(19) United States
(12) Patent Application Publication
(10) Pub. No.: US 2025/0049964 A1
(43) Pub. Date: Feb. 13, 2025

(54) INJECTABLE SHEAR-THINNING AND SELF-ASSEMBLING HYDROGELS

(71) Applicant: Boston Scientific Scimed, Inc., Maple Grove, MN (US)

(72) Inventors: Yen-Hao Hsu, Shrewsbury, MA (US); Nicolas Ball-Jones, Minneapolis, MN (US); Sasha Viola Rios, Minneapolis, MN (US); Joseph Thomas Delaney, JR., Minneapolis, MN (US); Cristian Parisi, Boston, MA (US)

(73) Assignee: Boston Scientific Scimed, Inc., Maple Grove, MN (US)

(21) Appl. No.: 18/796,777

(22) Filed: Aug. 7, 2024

Related U.S. Application Data
(60) Provisional application No. 63/518,165, filed on Aug. 8, 2023.

(51) Int. Cl. A61K 49/04

(52) U.S. Cl. CPC A61K 49/0442; A61K 49/0457

(57) ABSTRACT

In some aspects, the present disclosure pertains to an injectable hydrogel that comprises (a) one or more types of polymeric hydrogen bond donors, (b) one or more types of polymeric hydrogen bond acceptors and (c) water. Other aspects of the present disclosure pertain to kits that comprise a delivery device and one or more containers that contain an injectable hydrogel that comprises one or more types of polymeric hydrogen bond donors, one or more types of polymeric hydrogen bond acceptors, and water. Further aspects of the present disclosure pertain to medical procedures that comprise administering to a subject an injectable hydrogel that comprises one or more types of polymeric hydrogen bond donors, one or more types of polymeric hydrogen bond acceptors, and water.

### CROSS REFERENCE TO RELATED APPLICATIONS

This application claims the benefit of U.S. Provisional Patent Application Ser. No. 63/518,165 filed on Aug. 8, 2023, the disclosure of which is incorporated herein by reference.

### FIELD

The present disclosure relates to injectable shear-thinning and self-assembling hydrogels. The hydrogels are useful, for example, in various medical applications BACKGROUND

Hydrogels with shear-thinning properties are known which undergo a reversible gel-sol transition upon the application of shear stress. J. Pushpamalar, et al., “Development of a polysaccharide-based hydrogel drug delivery system (DDS): An update.” Gels, 2021, 7(4), p. 153. Shear-thinning hydrogels are increasingly used in drug delivery systems as a result of their ability to conform to the shape of an injection cavity, which maximizes contact with targeted tissue for localized drug delivery. Id. Hydrogels with shear-thinning properties have been reported to provide smooth injection without injection needle clogging, with the hydrogels returning to their original properties once mechanical load (shear stress) is removed. M. H. Chen, et al., “Methods to assess shear-thinning hydrogels for application as injectable biomaterials.” ACS Biomater. Sci. Eng. 2017, 3, 3146-3160. Hydrogels assembled by physical crosslinking of polyvinyl alcohol (PVA) and polyvinyl pyrrolidone (PVP) and containing doxorubicin have been noted to be able to deform under high shear and subsequently retain their original shape upon the removal of the high shear, demonstrating both shear-thinning and self-healing properties. N. K. Prasad, et al., “Discerning the self-healing, shear-thinning characteristics and therapeutic efficacy of hydrogel drug carriers migrating through constricted microchannel resembling blood microcapillary,” Colloids Surf A Physiochem. Eng. Asp. 2021, 626, 127070. FIG. 1 on page 5 of J. Pushpamalar, et al., schematically illustrates the shear-thinning and self-healing properties of a doxorubicin-loaded poly(vinyl alcohol)/poly(vinyl pyrrolidone) hydrogel. A shear-thinning hydrogel containing gelatin and laponite for localized drug delivery and further containing chitosan and poly(N-isopropylacrylamide-co-acrylic acid) particles to render the hydrogel pH-responsive has also been reported. S. Gharaie, et al., “Smart shear-thinning hydrogels as injectable drug delivery systems.” Polymers, 2018, 10(12), p. 1317.

The present disclosure provides additional shear-thinning hydrogels with unique properties as well as various novel applications for shear-thinning hydrogels beyond drug delivery.

### SUMMARY

In some aspects, the present disclosure provides an injectable hydrogel that comprises (a) one or more types of polymeric hydrogen bond donors, (b) one or more types of polymeric hydrogen bond acceptors and (c) water.

In some embodiments, which can be used in conjunction with the above aspects, the one or more types of polymeric hydrogen bond donors are selected from polyvinyl alcohol, poly(hydroxyalkyl acrylates), poly(hydroxyalkyl methacrylates), polyvinylphenol, and polysaccharides.

In some embodiments, which can be used in conjunction with the above aspects and embodiments, the one or more types of polymeric hydrogen bond acceptors are selected from polyvinyl pyrrolidone, poly(4-vinylpyridine), poly(ethylene oxide), polyacrylamide, polymethylmethacrylate, polycaprolactone, poly(vinyl acrylate) and poly(2-alkyl-2-oxazolines).

In some embodiments, which can be used in conjunction with the above aspects and embodiments, the one or more types of polymeric hydrogen bond donors and the one or more types of polymeric hydrogen bond acceptors range from 1 to 150 kDa in weight average molecular weight.

In some embodiments, which can be used in conjunction with the above aspects and embodiments, the one or more types of polymeric hydrogen bond donors or the one or more types of polymeric hydrogen bond acceptors are in the form of particles.

In some embodiments, which can be used in conjunction with the above aspects and embodiments, the injectable hydrogel comprises (a) 0.25 to 25 wt % of the one or more types of polymeric hydrogen bond donors, (b) 0.25 to 25 wt % of the one or more types of polymeric hydrogen bond acceptors and (c) 30 to 99 wt % of the water.

In some embodiments, which can be used in conjunction with the above aspects and embodiments, the injectable hydrogel further comprises one or types of radiopaque atoms.

In some embodiments, which can be used in conjunction with the above aspects and embodiments, the one or more types of polymeric hydrogen bond donors comprise an iodinated polymeric hydrogen bond donor and/or the one or more types of polymeric hydrogen bond acceptors comprise an iodinated polymeric hydrogen bond acceptor.

In some embodiments, the iodinated polymeric hydrogen bond donor and/or the iodinated polymeric hydrogen bond acceptor comprises covalently attached iodinated aromatic groups. In some of these embodiments, the covalently attached iodinated aromatic groups are interspersed with hydrogen bond donor groups along a backbone of the iodinated polymeric hydrogen bond donor or the covalently attached iodinated aromatic groups are provided in a first polymer block and the hydrogen bond donor groups are provided in a second polymer block of the iodinated polymeric hydrogen bond donor. In some of these embodiments, the covalently attached iodinated aromatic groups are interspersed with hydrogen bond acceptor groups along a backbone of the iodinated polymeric hydrogen bond acceptor or the covalently attached iodinated aromatic groups are provided in a first polymer block and the hydrogen bond acceptor groups are provided in a second polymer block of the iodinated polymeric hydrogen bond acceptor. In some of these embodiments, the covalently attached iodinated aromatic groups are linked to the iodinated polymeric hydrogen bond donor through ester or cyclic acetal linkages and/or the covalently attached iodinated aromatic groups are linked to the iodinated polymeric hydrogen bond acceptor through ester or cyclic acetal linkages.

In some embodiments, which can be used in conjunction with the above aspects and embodiments, the injectable hydrogel of has a radiopacity that is greater than 100 Hounsfield units.

In some embodiments, which can be used in conjunction with the above aspects and embodiments, the injectable hydrogel further comprises one or more additional agents selected from therapeutic agents, imaging agents, colorants, tonicity adjusting agents, suspension agents, wetting agents, and pH adjusting agents.

In some embodiments, which can be used in conjunction with the above aspects and embodiments, the injectable hydrogel is a sterile composition.

In some embodiments, which can be used in conjunction with the above aspects and embodiments, the injectable hydrogel is provided in a container. For example, the injectable hydrogel may be provided in a preloaded syringe.

In other aspects, the present disclosure pertains to kits that one or more containers that contain (a) an injectable hydrogel in accordance with any of the above aspects and embodiments and (b) a delivery device. In some embodiments, the delivery device comprises a syringe that is loaded with the injectable hydrogel with the above aspects and embodiments.

In other aspects, the present disclosure pertains to medical procedures comprising administering to a subject an injectable hydrogel in accordance with any of the above aspects and embodiments.

In some embodiments, the method comprises injecting the injectable hydrogel into the subject. For example, the injectable hydrogel may be administered by parenteral administration, may be administered using a catheter or a syringe, and/or may be administered under image guidance.

Potential benefits associated with the present disclosure include one or more of the following: hydrogels are provided that can be injected smoothly by a health care provider, the health care provider can pause injection without risk of needle clogging during product injection, and a single syringe can be used for injection.

Other benefits associated with the present disclosure include the ability transform a hydrogel having a first shape (e.g., the shape of a container such as a syringe barrel) into a viscous liquid during injection, after which the viscous liquid is transformed back into a hydrogel having a new shape that reflects the new environment. The ability of the viscous liquid to transform back into a hydrogel improves material retention and restores mechanical properties of the hydrogel. Such properties are useful, for example, in creating spacers for radiation and other cancer therapies and in injectable embolization applications.

The above and other aspects, embodiments, features and benefits of the present disclosure will be readily apparent from the following detailed description.

### BRIEF DESCRIPTION OF THE DRAWINGS

FIG. 1A schematically illustrates self-assembly of an injectable hydrogel from a polymeric hydrogen bond donor (polyhydroxyethylmethacrylate) and a polymeric hydrogen bond acceptor (polyvinylpyrrolidone), in accordance with an embodiment of the present disclosure.

FIG. 1B schematically illustrates shear-thinning (disassembly) and self-assembly properties of the hydrogel of FIG. 1A.

FIG. 2A schematically illustrates the synthesis of a radiopaque polymeric hydrogen bond donor (an iodinated polyhydroxyethylmethacrylate), in accordance with an embodiment of the present disclosure.

FIG. 2B schematically illustrates self-assembly of a hydrogel from the radiopaque polymeric hydrogen bond donor of FIG. 2A and a polymeric hydrogen bond acceptor (polyvinylpyrrolidone), in accordance with an embodiment of the present disclosure.

FIG. 3A schematically illustrates the synthesis of a radiopaque polymeric hydrogen bond donor (an iodinated polyvinyl alcohol), in accordance with another embodiment of the present disclosure.

FIG. 3B schematically illustrates self-assembly of a hydrogel from the radiopaque polymeric hydrogen bond donor of FIG. 3A and a polymeric hydrogen bond acceptor (polyvinylpyrrolidone), in accordance with an embodiment of the present disclosure.

FIG. 4 schematically illustrates a catheter and a syringe that is loaded with an injectable hydrogel, in accordance with an embodiment of the present disclosure.

### DETAILED DESCRIPTION

The present disclosure provides shear-thinning and self-assembling injectable hydrogels. The shear-thinning properties of such hydrogels allow for efficient injectability, as the hydrogels exhibit viscous flow under shear. The self-assembling properties of such hydrogels (also referred to as self-healing properties) allows for re-formation and stabilization of the hydrogel when the shear stress is removed. As used herein, self-assembly and self-healing refer to the spontaneous formation of new bonds within a material after old bonds within the material are broken. As used herein, a hydrogel refers to a water-containing three-dimensional network of crosslinked polymers.

In various embodiments, the injectable hydrogels of the present disclosure comprise (a) one or more types of hydrogen bond donors, (b) one or more types of hydrogen bond acceptors, and (c) water.

Such hydrogels comprise hydrogen-bond-based crosslinks which dissociate when a shear stress is applied and which spontaneously self-assemble when the shear stress is removed. Such disassociation may occur, for example, when a shear stress is applied during injection from a syringe. Upon dissociation of the hydrogen-bond-based crosslinks, the hydrogel becomes a viscous liquid that can be transported to a target site though a suitable delivery device, such as a tube (e.g. catheter/microcatheter) or a needle. Once delivered to the target site and the shear stress diminishes, the hydrogen bonds spontaneously re-associate (i.e., self-assemble), reforming the hydrogel at the target site. The transformation of the viscous liquid back into a hydrogel results in improved material retention and mechanical properties.

In some embodiments, the injectable hydrogels of the present disclosure exhibit yielding behavior. For example, after being subjected to a threshold yield strain, the injectable hydrogels may exhibit sharp decreases in storage and loss moduli, which decreases in moduli are recovered at low strains upon cessation of shear.

Hydrogen bond acceptors for use in the present disclosure include one or more types of polymeric hydrogen bond donors, examples of which include polyvinyl alcohol, poly(hydroxy-C₁-C₆-alkyl acrylates) such as poly(hydroxymethyl acrylate), poly(hydroxyethyl acrylate), poly(hydroxypropyl acrylate), poly(hydroxybutyl acrylate), etc., poly(hydroxy-C₁-C₆-alkyl methacrylates) such as poly(hydroxymethyl methacrylate), poly(hydroxyethyl methacrylate), poly(hydroxypropyl methacrylate), poly(hydroxybutyl methacrylate), etc., polyvinylphenol, polyacrylic acid, polysaccharides, and polystyrene sulfonic acid, poly(vinylphosphonic acid), poly(vinylmethylether), and poly(maleic acid).

Hydrogen bond acceptors for use in the present disclosure include one or more types of polymeric hydrogen bond acceptors, examples of which include polyvinyl pyrrolidone, poly(4-vinylpyridine), poly(ethylene oxide), polyacrylamide, polymethylmethacrylate, polycaprolactone, poly(vinyl acrylate), poly(2-C₁-C₆-alkyl-2-oxazolines) such as poly(2-methyl-2-oxazoline), poly(2-ethyl-2-oxazoline), poly(2-isopropyl-2-oxazoline), etc., and water-born polyurethanes.

The polymeric hydrogen bond acceptors or the polymeric hydrogen bond donors can be in particulate form, wherein a number of the polymer molecules are bound together, or in non-particulate form, wherein the polymer molecules not bound together and are disassociated or disassociable from one another.

For example, in some embodiments, the hydrogels of the present disclosure may contain a polymeric hydrogen bond acceptor in non-particulate form and a polymeric hydrogen bond donor in non-particulate form. In some embodiments, the hydrogels may contain a polymeric hydrogen bond acceptor in non-particulate form and a polymeric hydrogen bond donor in particulate form. In some embodiments, the hydrogels may contain a polymeric hydrogen bond acceptor in particulate form and a polymeric hydrogen bond donor in non-particulate form.

When the polymeric hydrogen bond acceptor or polymeric hydrogen bond donor is in particulate form, multiple polymeric hydrogen bond donor molecules or multiple polymeric hydrogen bond acceptor molecules are linked together such that they do not dissociate from one another under shear stresses associated with injection. For example, the polymeric hydrogen bond donor molecules or the polymeric hydrogen bond acceptor molecules may be held in particulate form by covalent crosslinking or by interpenetrating networks.

Particles of the polymeric hydrogen bond donor or the polymeric hydrogen bond acceptor may range may vary in size, for example, ranging from 1 nm to 300 microns, for example, ranging from 1 nm 3 nm to 10 nm to 30 nm to 100 nm to 300 nm to 1 micron to 3 microns to 10 microns to 30 microns to 100 microns to 300 microns (i.e., between any two of the preceding values) in largest cross-sectional dimension (e.g., diameter for a spherical particle, length for a rod-shaped particle, greatest width for a plate-shaped particle, etc.), and typically range from 50 nm to 1250 nm in size.

When the polymeric hydrogen bond acceptor or the polymeric hydrogen bond donor is in non-particulate form, the molecules of the polymeric hydrogen bond acceptor or the polymeric hydrogen bond donor are not linked to one another and are dissociable from one another under the shear stresses associated with injection.

Polymeric hydrogen bond donor molecules and polymeric hydrogen bond accepter molecules in accordance with the present disclosure may vary in length, for example, having weight average molecular weight (Mw) ranging from 2.5 kDa or less to 200 kDa or more, for example, ranging anywhere from 1 to kDa to 2.5 kDa to 5 kDa to 10 kDa to 25 kDa to 50 kDa to 100 kDa to 250 kDa (i.e., between any two of the preceding values), among other possible values. Polydispersity values for the molecules of the polymeric hydrogen bond acceptor and the polymeric hydrogen bond donor may vary but are typically less than 8, more typically less than 2, even more typically less than 1.5.

The concentration of the one or more types of hydrogen bond donors in the injectable hydrogels of the present disclosure may vary widely, but typically ranges from 0.25 wt % or less to 25 wt % or more, for example, ranging anywhere from 0.25 to 0.5 to 1 to 2.5 to 5 to 10 to 25 wt %. Similarly, the concentration of the one or more types of hydrogen bond acceptors in the injectable hydrogels of the present disclosure may vary widely, but typically ranges from 0.25 or less to 25 wt % or more, for example, ranging anywhere from 0.25 to 0.5 to 1 to 2.5 to 5 to 10 to 25 wt %.

The water in the injectable hydrogels of the present disclosure may be provided in the form of ultrapure water, water for injection, saline, phosphate buffered saline, or high-ion-content water.

In some embodiments, the injectable hydrogels of the present disclosure contain between 0.25 wt % or less and 30 wt % or more water, for example, ranging anywhere from 0.25 to 0.5 to 1 to 2.5 to 5 to 10 to 20 to 30 wt %.

In some embodiments, the injectable hydrogels of the present disclosure may further contain additional agents, examples of which are discussed further below.

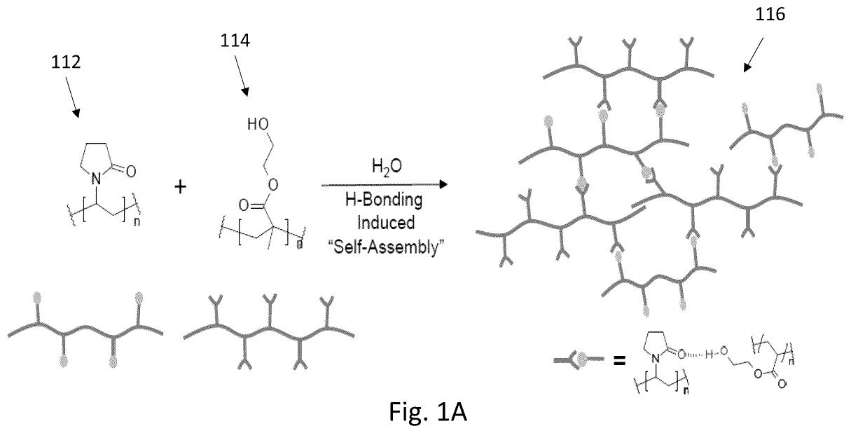

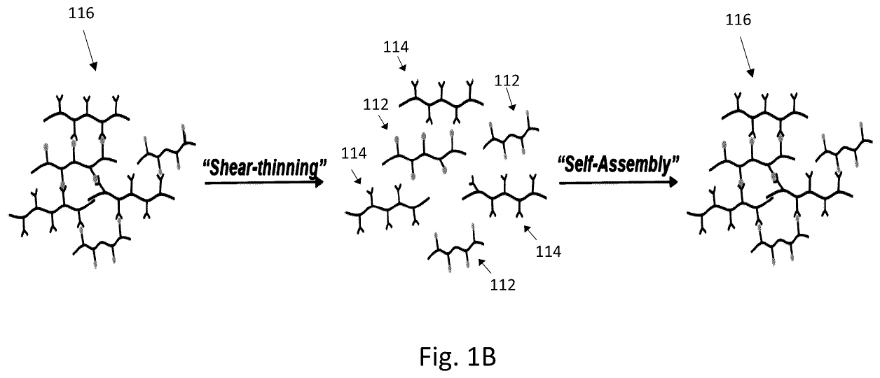

In one particular embodiment shown schematically in FIG. 1A, non-particulate polyvinyl pyrrolidone hydrogen bond acceptor molecules (112) and non-particulate poly(hydroxyalkyl acrylate) hydrogen bond donor molecules, specifically poly(2-hydroxyethyl methacrylate) hydrogen bond donor molecules (114), are combined, after which they assemble into a shear-thinning hydrogel (116) by hydrogen-bonding-induced self-assembly. In FIG. 1A, n is an integer, typically ranging from 20 or less to 1000 or more (e.g., ranging anywhere from 20 to 50 to 100 to 200 to 500 to 1000) for the polyvinyl pyrrolidone molecules (112) and typically ranging from 20 or less to 1000 or more (e.g., ranging anywhere from 20 to 50 to 100 to 200 to 500 to 1000) for the poly(2-hydroxyethyl methacrylate) molecules (114),

As shown schematically in FIG. 1B, when subjected to mechanical shear, the shear-thinning hydrogel (116) disassembles into individual polyvinyl pyrrolidone molecules (112) and poly(2-hydroxyethyl methacrylate) molecules (114). Then, when mechanical shear conditions are discontinued, the individual polyvinyl pyrrolidone molecules (112) and poly(2-hydroxyethyl methacrylate) molecules (114) reassemble into a shear-thinning hydrogel (116).

As a result of this behavior, an injectable hydrogel is provided which can undergo disassembly (shear-thinning) during injection through a syringe, thereby providing smooth injection, and then re-assemble at a target site when the shear forces associated with the injection are removed. In addition, the shape of the hydrogel is transformed during injection and a new shape is established at the target injection site due to the self-assembling characteristics of the hydrogel.

In some embodiments, the injectable hydrogels of the present disclosure further comprises one or more radiopaque atoms, which may be selected, for example, from Br, I, Bi, Ba, Gd, Ta, Zn, W and Au.

In some embodiments, the one or more radiopaque atoms are iodine atoms, which may be provided in a covalently attached iodinated moiety. In some of these embodiments, the iodinated moiety comprises an iodinated aromatic group. Examples of iodinated aromatic groups include iodine-substituted monocyclic aromatic groups and iodine-substituted multicyclic aromatic groups, such as iodinated phenyl groups, iodinated naphthyl groups, iodinated anthracenyl groups, iodinated phenanthrenyl groups, or iodinated tetracenyl groups. The iodinated aromatic groups may be substituted with one, two, three, four, five, six, or more iodine atoms. In some of these embodiments, the aromatic groups may be further substituted with one or more hydrophilic groups, for example, one, two, three, four, five, six or more hydrophilic groups. The hydrophilic groups may be hydroxyl-containing groups, which may be selected, for example, from hydroxyl groups and hydroxyalkyl groups (e.g., hydroxyalkyl groups containing one carbon, two carbons, three carbons, four carbons, etc.), amide-containing groups (e.g., amide groups containing two carbons, three carbons, four carbons, five carbons, etc.), and sulfonamide groups, among others. The hydrophilic groups may be linked to the monocyclic or multicyclic aromatic structures directly or through any suitable linking moiety, which may be selected, for example, from ether groups, ester groups, amide groups, amine groups, or carbonate groups, among others.

In one exemplary embodiment, hydroxyl groups of a precursor polymeric hydrogen bond donor are reacted with carboxy groups of radiopaque precursor compound in an ester coupling reaction. This reaction step forms a radiopaque polymeric hydrogen bond donor that comprises a plurality of hydrogen-bond-donating hydroxyl groups and a plurality of radiopaque groups interspersed along the backbone of the radiopaque polymeric hydrogen bond donor.

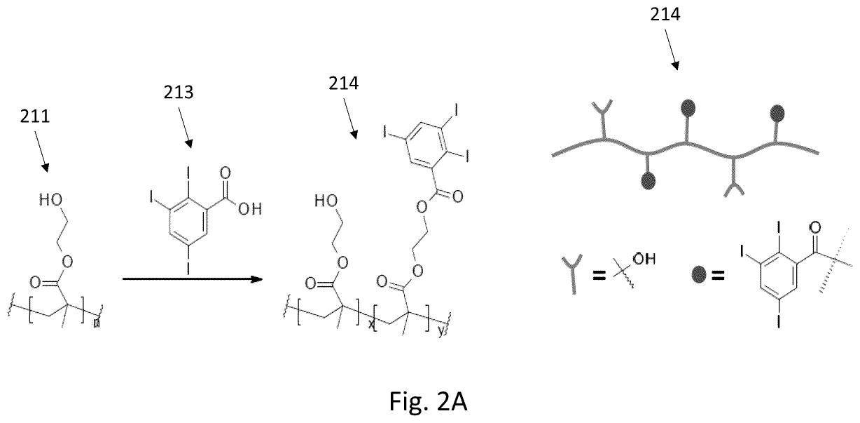

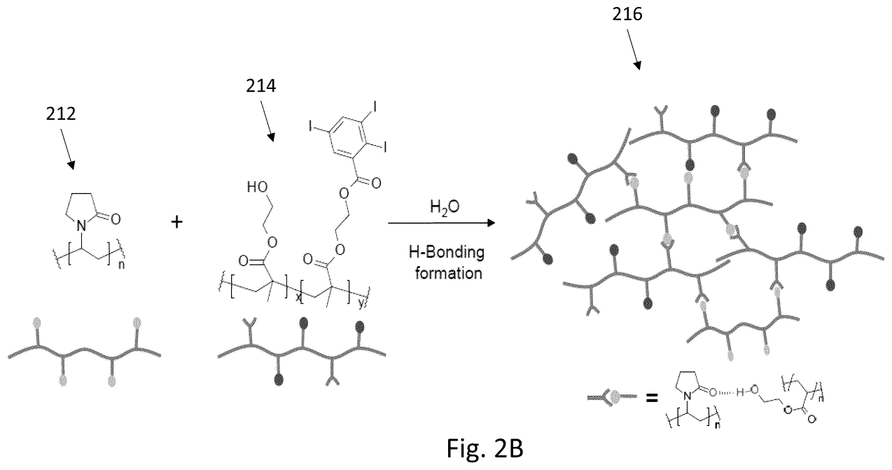

In a particular example shown in FIG. 2A, hydroxyl groups of a precursor polymeric hydrogen bond donor, specifically, poly(hydroxyethyl methacrylate) (211), where n is an integer, for example, ranging from 20 to 1000, are reacted with a carboxyl group of a radiopaque precursor compound, specifically, 2,3,5-triiodobenzoic acid (213), thereby forming a radiopaque polymeric hydrogen bond donor, specifically, an iodine-substituted poly(hydroxyethyl methacrylate) (214) in which 2,3,5-triiodobenzene moieties are attached to the poly(hydroxyethyl methacrylate) backbone through ester linkages. Such an ester-coupling reaction may be performed using a suitable coupling reagent, for instance, a carbodiimide coupling reagent such as dicyclohexylcarbodiimide (DCC) or diisopropylcarbodiimide (DIC). In FIG. 2A, x and y are integers representing the numbers of non-substituted hydroxyl monomers and iodine substituted monomers, respectively, in the iodine-substituted poly(hydroxyethyl methacrylate) (214). It is noted that the ratio of the integers x and y in the iodine-substituted poly(hydroxyethyl methacrylate) (214) are tunable with greater amounts of x providing greater numbers of hydrogen bond donors and with greater amounts of y providing greater radiopacity. Typically x will range from 60 to 99% of the value of n in the precursor poly(hydroxyethyl methacrylate) (211), while y will typically range from 1 to 40% of the value of n in the precursor poly(hydroxyethyl methacrylate) (211).

Although poly(2-hydroxyethyl methacrylate) is used as a precursor polymeric hydrogen bond donor in FIG. 2A, other hydroxylated polymers, including other poly(hydroxyalkyl methacrylates), poly(hydroxyalkyl acrylates), polyvinyl alcohol, polyvinylphenol, or polysaccharides, may be used as precursor polymeric hydrogen bond donors.

Moreover, although triiodobenzoic acid is used as a radiopaque precursor compound in FIG. 2A, other radiopaque precursor compounds may be employed, including

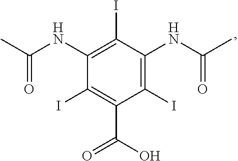 [SMILES: CC(=O)Nc1c(I)c(NC(C)=O)c(I)c(C(=O)O)c1I]

diatrizoic acid, CAS #117-96-4,

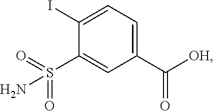 [SMILES: NS(=O)(=O)c1cc(C(=O)O)ccc1I]

5-formyl-2-iodobenzen sulfonamide, CAS #1289167-85-6,

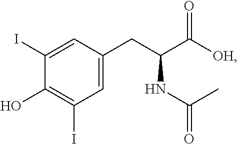 [SMILES: CC(=O)N[C@@H](Cc1cc(I)c(O)c(I)c1)C(=O)O]

N-acetyl-3,5-diiodo-L-tyrosine, CAS #1027-28-7,

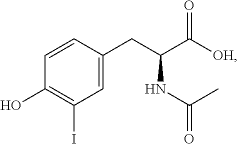 [SMILES: CC(=O)N[C@@H](Cc1ccc(O)c(I)c1)C(=O)O]

N-acetyl-3-diiodo-L-tyrosine, CAS #1023-47-8, and

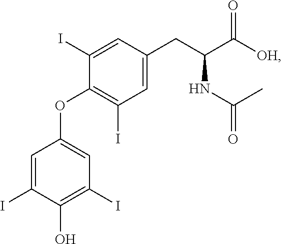 [SMILES: CC(=O)N[C@@H](Cc1cc(I)c(Oc2cc(I)c(O)c(I)c2)c(I)c1)C(=O)O]

N-acetyl-thyroxine, CAS #26041-51-0, among many others.

In a particular embodiment shown schematically in FIG. 2B, polyvinyl pyrrolidone molecules (212) and iodine-substituted poly(2-hydroxyethyl methacrylate) molecules (214), are combined, after which they self-assemble into a shear-thinning hydrogel (216) by hydrogen-bonding-induced self-assembly.

In another exemplary embodiment, a portion of the diol groups of a precursor polymeric hydrogen bond donor having 1,2 diol groups or 1,3 diol groups are reacted with aldehyde groups of radiopaque precursor compound in a cyclic acetal coupling reaction, generally under acidic conditions. This reaction step forms a radiopaque polymeric hydrogen bond donor that comprises a plurality of hydrogen-bond-donating hydroxyl groups and a plurality of radiopaque groups that are covalently linked to the backbone of the polymeric hydrogen bond donor through cyclic acetal groups.

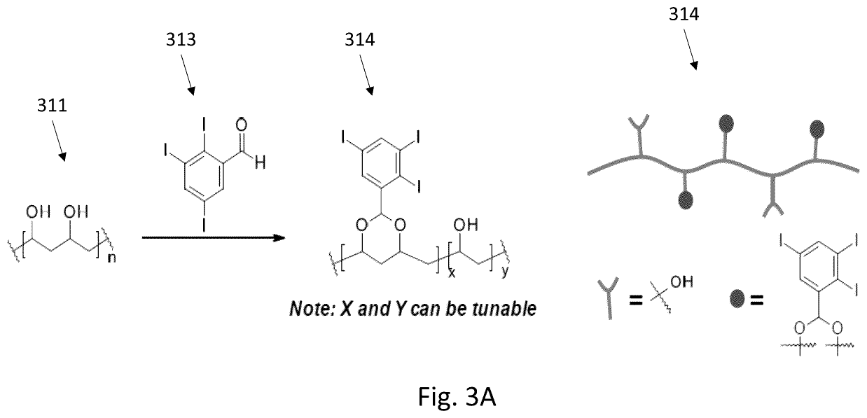

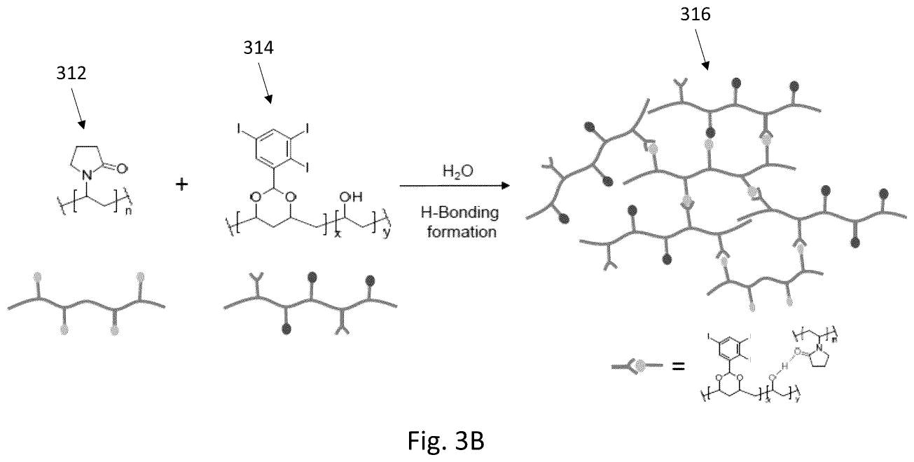

In a particular example shown in FIG. 3A, 1,3 diol groups of a precursor polymeric hydrogen bond donor, specifically, poly(vinyl alcohol) (311), where n may range, for example, from 20 to 1500, are reacted with the aldehyde group of a radiopaque precursor compound, specifically, 2,3,5-triiodobenzaldehyde (313) thereby forming a radiopaque polymeric hydrogen bond donor, specifically, an iodine-substituted poly(vinyl alcohol) (314) in which 2,3,5-triiodobenzene moieties are attached to the poly(vinyl alcohol) backbone through cyclic acetal linkages. Such a coupling reaction is generally performed under acidic conditions. It is noted that the ratio of the integers x and y in the iodine-substituted poly(vinyl alcohol) (314) are tunable with greater amounts of x providing greater numbers of hydrogen bond donors and with greater amounts of y providing greater radiopacity. Typically x will range from 1 to 40% of the value of n in the precursor poly(vinyl alcohol) (311), while y will typically range from 60 to 99% of the value of n in the precursor poly(vinyl alcohol) (311). Although polyvinyl alcohol is used as a precursor polymeric hydrogen bond donor in FIG. 3A, other diols, including ethylene vinyl alcohol, or glycerin-2-acrylate may be used as precursor polymeric hydrogen bond donors. Moreover, although 2,3,5-triiodobenzaldehyde is used as a radiopaque precursor compound in FIG. 3A, other aldehydes, including 2,3,5-triiodobenzaldehyde, 2,3,4,6-tetraiodobenzyaldehyde and 2-(2,4,6-triiodophenoxy)acetaldehyde, may be employed as radiopaque precursor compounds.

In a particular embodiment shown schematically in FIG. 3B, polyvinyl pyrrolidone molecules (312) and iodine-substituted poly(vinyl alcohol) molecules (314), are combined, after which they self-assemble into a shear-thinning hydrogel (316) by hydrogen-bonding-induced self-assembly.

In some embodiments, iodinated block copolymers are provided which comprise a polymeric hydrogen bond donor block, for example, selected from the above polymeric hydrogen bond donors, and an iodinated polymer block. In some embodiments, iodinated block copolymers are provided which comprise a polymeric hydrogen bond acceptor block, for example, selected from the above polymeric hydrogen bond acceptors, and an iodinated polymer block. Examples of iodinated polymer blocks include iodinated polystyrene blocks. In these embodiments, the polymeric hydrogen bond donor blocks may range, for example, from 20 to 1500 monomers in length, the polymeric hydrogen bond acceptor blocks may range, for example, from 20 to 1500 monomers in length, and the iodinated polymer blocks may range, for example, from 1 to 50 monomers in length.

In various embodiments, the hydrogels of the present disclosure are visible under fluoroscopy. In various embodiments, the hydrogels have a radiopacity that is greater than 100 Hounsfield units (HU), beneficially anywhere ranging from 100 HU to 250 HU to 500 HU to 750 HU to 1000 HU to 2000 HU or more (in other words, ranging between any two of the preceding numerical values).

The injectable hydrogels of the present disclosure may be formed using a variety of methods. The one or more types of hydrogen bond donors, the one or more types of hydrogen bond acceptors and the water may be mixed in any order. For example, the one or more types of hydrogen bond donors and the one or more types of hydrogen bond acceptors may be first mixed and then combined with the water. As another example, the one or more types of hydrogen bond donors and the water may be first mixed and then combined with the one or more types of hydrogen bond acceptors. As another example, the one or more types of hydrogen bond acceptors and the water may be first mixed and then combined with the one or more types of hydrogen bond donors. As another example, a first composition containing the one or more types of hydrogen bond donors and water may be formed, a second composition containing the one or more types of hydrogen bond acceptors and water may be formed, and the first and second compositions then combined. As yet another example, the one or more types of hydrogen bond acceptors, the one or more types of hydrogen bond donors and the water may be simultaneously mixed. Mixing may be performed by any suitable mixing technique, including, for example, centrifugal mixing, manual mixing, high shear dispersing, vacuum mixing, vortexing, and/or syringe-to-syringe mixing.

The compositions of the present disclosure may be sterilized using any suitable method such as heat (e.g., dry heat, moist heat, etc.), sterile filtration, supercritical CO₂, gamma-ray irradiation, x-ray irradiation or electron beam irradiation. The compositions of the present disclosure can be sterilized in hydrogel form or in powder form (to which a sterile liquid such as water, saline, etc. may be added). The compositions may be sterilized while inside a reservoir, such as a syringe barrel, vial, or ampule.

In various embodiments, the injectable hydrogels of the present disclosure contain one or more agents in addition to the one or more types of hydrogen bond donors, the one or more types of hydrogen bond acceptors and the water. Examples of such additional agents include therapeutic agents, imaging agents, colorants, tonicity adjusting agents, suspension agents, wetting agents, and pH adjusting agents.

Examples of therapeutic agents include antithrombotic agents, anticoagulant agents, antiplatelet agents, thrombolytic agents, antibodies, anti-cancer drugs, antiproliferative agents, anti-inflammatory agents, hyperplasia inhibiting agents, anti-restenosis agents, steroids, anti-allergic agents, hemostatic agents, smooth muscle cell inhibitors, antibiotics, antimicrobials, anti-fungal agents, analgesics, anesthetics, immunosuppressants, growth factors, growth factor inhibitors, cell adhesion inhibitors, cell adhesion promoters, anti-angiogenic agents, cytotoxic agents, chemotherapeutic agents, checkpoint inhibitors, immune modulatory cytokines, T-cell agonists, and STING (stimulator of interferon genes) agonists, among others.

Examples of imaging agents include (a) fluorescent dyes such as fluorescein, indocyanine green, or fluorescent proteins (e.g. green, blue, cyan fluorescent proteins), (b) contrast agents for use in conjunction with magnetic resonance imaging (MRI), including contrast agents that contain elements that form paramagnetic ions, such as Gd^{(III)}, Mn^{(II)}, Fe^{(III)} and compounds (including chelates) containing the same, such as gadolinium ion chelated with diethylenetriaminepentaacetic acid, (c) contrast agents for use in conjunction with ultrasound imaging, including organic and inorganic echogenic particles (i.e., particles that result in an increase in the reflected ultrasonic energy) or organic and inorganic echolucent particles (i.e., particles that result in a decrease in the reflected ultrasonic energy), (d) contrast agents for use in connection with near-infrared (NIR) imaging, which can be selected to impart near-infrared fluorescence to the injectable hydrogels of the present disclosure, allowing for deep tissue imaging and device marking, for instance, NIR-sensitive nanoparticles such as gold nanoshells, carbon nanotubes (e.g., nanotubes derivatized with hydroxyl or carboxyl groups, for instance, partially oxidized carbon nanotubes), dye-containing nanoparticles, such as dye-doped nanofibers and dye-encapsulating nanoparticles, and semiconductor quantum dots, among others, and NIR-sensitive dyes such as cyanine dyes, squaraines, phthalocyanines, porphyrin derivatives and boron dipyrromethane (BODIPY) analogs, among others, (e) imagable radioisotopes including ^{99m}Tc, ²⁰¹Th, ⁵¹Cr, ⁶⁷Ga, ⁶⁸Ga, ¹¹¹In, ⁶⁴Cu, ⁸⁹Zr, ⁵⁹Fe, ⁴²K, ⁸²Rb, ²⁴Na, ⁴⁵Ti, ⁴⁴Sc, ⁵¹Cr and ¹⁷⁷Lu, among others, and (f) radiocontrast agents such as metallic particles, for example, particles of tantalum, tungsten, rhenium, niobium, molybdenum, and their alloys, which metallic particles may be spherical or non-spherical. Additional examples of radiocontrast agents include non-ionic radiocontrast agents, such as iohexol, iodixanol, ioversol, iopamidol, ioxilan, or iopromide, ionic radiocontrast agents such as diatrizoate, iothalamate, metrizoate, or ioxaglate, and iodinated oils, including ethiodized poppyseed oil (available as Lipiodol®).

Examples of colorants include brilliant blue (e.g., Brilliant Blue FCF, also known as FD&C Blue 1), indigo carmine (also known as FD&C Blue 2), indigo carmine lake, FD&C Blue 1 lake, and methylene blue (also known as methylthioninium chloride), among others.

Examples of additional agents further include tonicity adjusting agents such as sugars (e.g., dextrose, lactose, etc.), polyhydric alcohols (e.g., glycerol, propylene glycol, mannitol, sorbitol, etc.) and inorganic salts (e.g., potassium chloride, sodium chloride, etc.), among others, suspension agents including various surfactants, wetting agents, and polymers (e.g., albumen, PEO, polyvinyl alcohol, block copolymers, etc.), among others, and pH adjusting agents including various buffer solutes.

The injectable hydrogels of the present disclosure may be stored and transported in a sterile form. The injectable hydrogels may be shipped, for example, in a syringe, catheter, vial, ampoule, or other container.

In various embodiments, kits are provided, which may include one or more containers of injectable hydrogels as described herein as well other components. For example, the kits may include one or more delivery devices for delivering the injectable hydrogels to a subject such as syringes, catheters or tubing sets. In some embodiments, the kits may comprise an injectable hydrogel as described herein preloaded in a catheter and/or a syringe barrel and/or in a container such as a vial or ampule. Alternatively or in addition, kits may be provided that include one or more accessory devices such as guidewires. Alternatively or in addition, the kits may be provided that include one or more containers of liquid materials (e.g. contrast agent, sterile water for injection, physiological saline, phosphate buffer, etc.). Alternatively or in addition, the kits may further comprise an additional therapeutic agent, which may be selected, for example, from those described above, among others. Instructions, either as inserts or as labels, indicating quantities of the composition to be administered and/or guidelines for administration can also be included in the kits provided herein. In some embodiments, the instructions comprise instructions for performing one or more of the methods provided herein.

The injectable hydrogels described herein can be administered by a variety of routes, depending upon the desired medical outcome. In some embodiments, the administering comprises injecting the injectable hydrogel. In some embodiments, the injectable hydrogels are administered by parenteral administration. Parenteral administration includes intravenous, intraarterial, subcutaneous, intraperitoneal, or intramuscular injection or infusion. In some embodiments, the administering comprises an image guided procedure where computed tomography, fluoroscopy or ultrasound imaging is used to deliver the composition. In some embodiments, the administering comprises injecting the injectable hydrogel into the vascular system of a subject. In some embodiments, the administering comprises injecting the injectable hydrogel into a tumor of the subject or the vasculature supplying a tumor of the subject. In some embodiments, the administering is performed using a catheter or a syringe.

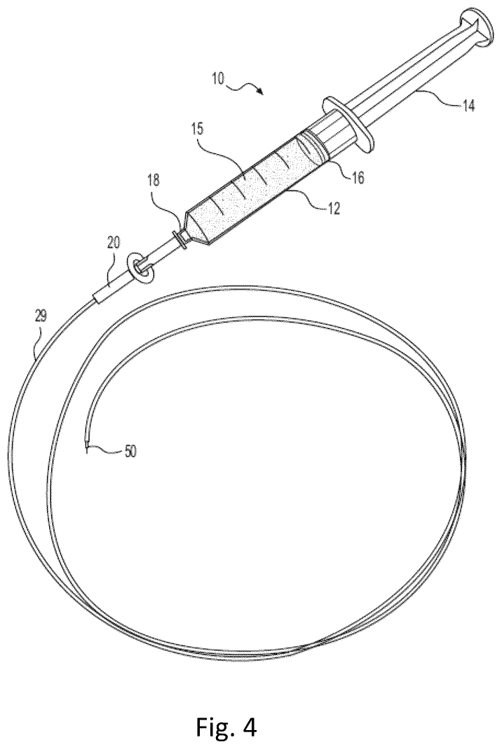

FIG. 4 illustrates an exemplary syringe 10 providing a reservoir for an injectable hydrogel described herein. The syringe 10 may comprise a barrel 12, a plunger 14, and one or more stoppers 16. The barrel 12 may include a Luer adapter (or other suitable adapter/connector), e.g., at the distal end 18 of the barrel 12, for attachment to an injection needle 50 via a flexible catheter 29. The proximal end of the catheter 29 may include a suitable connection 20 for receiving the barrel 12. In other examples, the barrel 12 may be directly coupled to the injection needle 50. The syringe barrel 12 may serve as a reservoir, containing an injectable hydrogel 15 for injection through the needle 50.

The injectable hydrogels described herein can be administered to patients for achieving a number of medical outcomes.

The injectable hydrogels described herein can be visualized (e.g., within a mammal) using any appropriate method during and/or after administration. For example, imaging techniques such as ultrasound, computed tomography, magnetic resonance imaging, and/or fluoroscopy can be used to visualize the injectable hydrogels provided herein.

The injectable hydrogels of the present disclosure may be used in a variety of medical procedures, including the following, among others: a procedure to inject the injectable hydrogel into a feeder artery to embolize tissue, including benign tumors, malignant tumors and other abnormal tissue, a procedure to introduce the injectable hydrogel between a first tissue and a second tissue to space the first tissue from the second tissue, a procedure to implant a fiducial marker comprising the injectable hydrogel (e.g., in the form of blebs), a procedure to implant a tissue regeneration scaffold comprising the injectable hydrogel, a procedure to implant a tissue support comprising the injectable hydrogel, a procedure to implant a tissue bulking agent comprising the injectable hydrogel, a procedure to implant a therapeutic-agent-containing depot comprising the injectable hydrogel, a tissue augmentation procedure comprising implanting the injectable hydrogel, or a procedure to control bleeding.

The injectable hydrogels can be injected for tissue augmentation or regeneration, the injectable hydrogels can be injected as a filler or replacement for soft tissue, the injectable hydrogels can be injected to provide mechanical support for compromised tissue, the injectable hydrogels can be injected as a scaffold, and/or the injectable hydrogels can be injected as a carrier of therapeutic agents in the treatment of diseases and cancers and the repair and regeneration of tissue, among other uses.

The injectable hydrogels may be injected in conjunction with a variety of medical procedures including the following: injection between the prostate or vagina and the rectum for spacing in radiation therapy for rectal cancer, injection between the rectum and the prostate for spacing in radiation therapy for prostate cancer, subcutaneous injection for palliative treatment of prostate cancer, transurethral or submucosal injection for female stress urinary incontinence, intra-vesical injection for urinary incontinence, uterine cavity injection for Asherman's syndrome, submucosal injection for anal incontinence, percutaneous injection for heart failure, intra-myocardial injection for heart failure and dilated cardiomyopathy, trans-endocardial injection for myocardial infarction, intra-articular injection for osteoarthritis, spinal injection for spinal fusion, and spine, oral-maxillofacial and orthopedic trauma surgeries, spinal injection for posterolateral lumbar spinal fusion, intra-discal injection for degenerative disc disease, injection between pancreas and duodenum for imaging of pancreatic adenocarcinoma, resection bed injection for imaging of oropharyngeal cancer, injection around circumference of tumor bed for imaging of bladder carcinoma, submucosal injection for gastroenterological tumors and polyps, visceral pleura injection for lung biopsy, kidney injection for type 2 diabetes and chronic kidney disease, renal cortex injection for chronic kidney disease from congenital anomalies of kidney and urinary tract, intra-vitreal injection for neovascular age-related macular degeneration, intra-tympanic injection for sensorineural hearing loss, dermis injection for correction of wrinkles, creases and folds, signs of facial fat loss, volume loss, shallow to deep contour deficiencies, correction of depressed cutaneous scars, perioral rhytids, lip augmentation, facial lipoatrophy, stimulation of natural collagen production.

The injectable hydrogels may be injected for the permanent or temporary occlusion of blood vessels, and thus may be useful for managing various diseases and conditions. For example, the injectable hydrogels may be used for the controlled, selective obliteration of the blood supply to benign and malignant tumors including treating solid tumors such as renal carcinoma, bone cancer, brain cancer, liver cancer, breast cancer, prostate cancer, benign prostatic hyperplasia, esophageal cancer, colon cancer, endometrial cancer, bladder cancer, cancer of the uterus, uterine fibroids (leiomyoma), cancer of the ovary, lung cancer, sarcoma, pancreatic cancer, and stomach cancer. The idea behind this treatment is that the flow of blood, which supplies nutrients to the tumor, will be blocked causing it to shrink. Embolization may be conducted as an enhancement to chemotherapy or radiation therapy. Treatment may be enhanced in the present disclosure by including a therapeutic agent (e.g., antineoplastic/antiproliferative/anti-miotic agent, toxin, ablation agent, etc.) in the injectable hydrogel.

Injectable hydrogels in accordance with the present disclosure may also be used to treat various other diseases, conditions and disorders, including treatment of the following: arteriovenous fistulas and malformations including, for example, aneurysms such as neurovascular and aortic aneurysms, pulmonary artery pseudoaneurysms, intracerebral arteriovenous fistula, cavernous sinus dural arteriovenous fistula and arterioportal fistula, varices, chronic venous insufficiency, varicocele, abscesses, pelvic congestion syndrome, gastrointestinal bleeding, renal bleeding, urinary bleeding, varicose bleeding, venous congestion disorder, hemorrhage, including uterine hemorrhage, and severe bleeding from the nose (epistaxis), as well as preoperative embolization (to reduce the amount of bleeding during a surgical procedure) and occlusion of saphenous vein side branches in a saphenous bypass graft procedure, among other uses. As elsewhere herein, treatment may be enhanced in the present disclosure by including a therapeutic agent in the particulate composition.

Injectable hydrogels in accordance with the present disclosure may be used further in tissue bulking applications, for example, as augmentative materials in the treatment of urinary incontinence, vesicourethral reflux, fecal incontinence, intrinsic sphincter deficiency (ISD) or gastro-esophageal reflux disease, or as augmentative materials for aesthetic improvement. For instance, a common method for treating patients with urinary incontinence is via periurethral or transperineal injection of a bulking material. In this regard, methods of injecting bulking agents commonly require the placement of a needle at a treatment region, for example, periurethrally or transperineally. The bulking agent is injected into a plurality of locations, assisted by visual aids, causing the urethral lining to coapt. In some cases, additional applications of bulking agent may be required. Treatment may be enhanced by including a therapeutic agent (e.g., proinflammatory agents, sclerosing agents, etc.) in the injectable hydrogel.

Injectable hydrogels in accordance with the present disclosure may be used in hemostasis, for example, by direct application to a bleeding site or injection into blood vessel leading to a bleeding site.

Injectable hydrogels in accordance with the present disclosure may be injected into a left atrial appendage during a left atrial appendage closure procedure. In some embodiments, the injectable hydrogels may be injected into the left atrial appendage after the introduction of a closure device such as the Watchman® left atrial appendage closure device available from Boston Scientific Corporation.

Injectable hydrogels in accordance with the present disclosure may be used in the treatment of aneurisms. For example, the injectable hydrogels may be introduced into an aneurism, either alone or with an embolic device such as an embolic coil or a liquid embolic.

Although various embodiments are specifically illustrated and described herein, it will be appreciated that modifications and variations of the present disclosure are covered by the above teachings and are within the purview of any appended claims without departing from the spirit and intended scope of the present disclosure.

1. An injectable hydrogel comprising (a) one or more types of polymeric hydrogen bond donors, (b) one or more types of polymeric hydrogen bond acceptors and (c) water.

2. The injectable hydrogel of claim 1, wherein the one or more types of polymeric hydrogen bond donors are selected from polyvinyl alcohol, poly(hydroxyalkyl acrylates), poly(hydroxyalkyl methacrylates), polyvinylphenol, and polysaccharides.

3. The injectable hydrogel of claim 1, wherein the one or more types of polymeric hydrogen bond acceptors are selected from polyvinyl pyrrolidone, poly(4-vinylpyridine), poly(ethylene oxide), polyacrylamide, polymethylmethacrylate, polycaprolactone, poly(vinyl acrylate) and poly(2-alkyl-2-oxazolines).

4. The injectable hydrogel of claim 1, wherein the one or more types of polymeric hydrogen bond donors or the one or more types of polymeric hydrogen bond acceptors are in the form of particles.

5. The injectable hydrogel of claim 1, comprising (a) 0.25 to 25 wt % of the one or more types of polymeric hydrogen bond donors, (b) 0.25 to 25 wt % of the one or more types of polymeric hydrogen bond acceptors and (c) 30 to 99 wt % of the water.

6. The injectable hydrogel of claim 1, further comprising one or types of radiopaque atoms.

7. The injectable hydrogel of claim 1, wherein the one or more types of polymeric hydrogen bond donors comprise an iodinated polymeric hydrogen bond donor and/or wherein the one or more types of polymeric hydrogen bond acceptors comprise an iodinated polymeric hydrogen bond acceptor.

8. The injectable hydrogel of claim 7, wherein the iodinated polymeric hydrogen bond donor and/or the iodinated polymeric hydrogen bond acceptor comprises covalently attached iodinated aromatic groups.

9. The injectable hydrogel of claim 8, (a) wherein the covalently attached iodinated aromatic groups are interspersed with hydrogen bond donor groups along a backbone of the iodinated polymeric hydrogen bond donor or the covalently attached iodinated aromatic groups are provided in a first polymer block and the hydrogen bond donor groups are provided in a second polymer block of the iodinated polymeric hydrogen bond donor and/or (b) wherein the covalently attached iodinated aromatic groups are interspersed with hydrogen bond acceptor groups along a backbone of the iodinated polymeric hydrogen bond acceptor or the covalently attached iodinated aromatic groups are provided in a first polymer block and the hydrogen bond acceptor groups are provided in a second polymer block of the iodinated polymeric hydrogen bond acceptor.

10. The injectable hydrogel of claim 8, wherein the covalently attached iodinated aromatic groups are linked to the iodinated polymeric hydrogen bond donor through ester or cyclic acetal linkages and/or wherein the covalently attached iodinated aromatic groups are linked to the iodinated polymeric hydrogen bond acceptor through ester or cyclic acetal linkages.

11. The injectable hydrogel of claim 6, wherein the injectable hydrogel has a radiopacity that is greater than 100 Hounsfield units.

12. The injectable hydrogel of claim 1, wherein the injectable hydrogel further comprises one or more additional agents selected from therapeutic agents, imaging agents, colorants, tonicity adjusting agents, suspension agents, wetting agents, and pH adjusting agents.

13. The injectable hydrogel of claim 1, wherein the injectable hydrogel is provided in a preloaded syringe.

14. A kit comprising (a) an injectable hydrogel comprising one or more types of polymeric hydrogen bond donors, one or more types of polymeric hydrogen bond acceptors, and water and (b) a delivery device.

15. The kit of claim 14, wherein the delivery device comprises a syringe that is loaded with the injectable hydrogel.

16. A medical procedure comprising administering to a subject an injectable hydrogel that comprises one or more types of polymeric hydrogen bond donors, one or more types of polymeric hydrogen bond acceptors, and water.

17. The medical procedure of claim 16, wherein the method comprises injecting the injectable hydrogel into the subject.

18. The medical procedure of claim 16, wherein the administering comprises parenteral administration.

19. The medical procedure of claim 16, wherein the administering is performed using a catheter or a syringe.

20. The medical procedure of claim 16, wherein the administering is performed under image guidance.
